(19) United States
(12) Patent Application Publication
(10) Pub. No.: US 2006/0067844 A1
(43) Pub. Date: Mar. 30, 2006

(54) Piston compressor, particularly hermetical refrigerant compressor

(75) Inventor: Frank Holm Iversen, Padborg (DK)

Correspondence Address: MCCORMICK, PAULDING & HUBER LLP, CITY PLACE II, 185 ASYLUM STREET, HARTFORD, CT, 06103, US

(73) Assignee: Danfoss Compressors GmbH, Flensburg (DE)

(21) Appl. No.: 11/235,851

(22) Filed: Sep. 27, 2005

(30) Foreign Application Priority Data
Sep. 29, 2004 (DE) 10 2004 047 159.2

(51) Int. Cl. A61M 1/00; F04B 53/00

(52) U.S. Cl. 417/458; 417/437

(57) ABSTRACT

The invention concerns a piston compressor (1), particularly a hermetical refrigerant compressor, with a compression chamber (2), which is bordered by a cylinder (3), a piston (4) and a valve plate arrangement (5), which has a suction valve arrangement (14, 15) and a pressure valve arrangement (16, 17). It is endeavoured to provide a piston compressor with a good efficiency. For this purpose, it is ensured that the suction valve arrangement (14, 15) opens into the compression chamber (2) in a central area of the cylinder (3) and the pressure valve arrangement (16, 17) has a plurality of pressure openings (16), which are located in an annular area surrounding the suction valve arrangement (14, 15).

### CROSS-REFERENCE TO RELATED APPLICATIONS

Applicant hereby claims foreign priority benefits under U.S.C. § 119 from German Patent Application No. 10 2004 047 159.2 filed on Sep. 29, 2004, the contents of which are incorporated by reference herein.

### FIELD OF THE INVENTION

The invention concerns a piston compressor, particularly a hermetical refrigerant compressor, with a compression chamber, which is bordered by a cylinder, a piston and a valve plate arrangement, which has a suction valve arrangement and a pressure valve arrangement.

### BACKGROUND OF THE INVENTION

In the following, the invention will be described by way of a refrigerant compressor as an example of a piston compressor. However, it can also be used with other piston compressors.

In a refrigerant compressor the compression chamber expands, when the piston is moved away from the valve plate arrangement. In this case, refrigerant gas is sucked into the compression chamber through the suction valve arrangement. When the piston moves in the opposite direction, that is, in the direction of the valve plate-arrangement, the refrigerant gas is first compressed and then discharged through the pressure valve arrangement.

From DE 35 26 255 A1 is known a piston compressor with a combined suction and pressure valve, in which a central pressure opening in a valve plate is covered by a valve element in the form of a lamella. Around the central pressure opening are located several suction openings, which a covered by a continuous valve element, which has the form of an circular disc. This circular disc is fixed on the valve plate by the pressure valve element. This design gives a considerable dead space in the upper dead end of the piston and a relatively large heating of the suction gas, as this flows into the compression chamber directly along the cylinder wall.

DE 27 26 089 A1 shows a valve plate for a piston compressor, in which both the suction valve arrangement and the pressure valve arrangement have several openings provided in kidney-shaped openings. The suction openings are arranged in an annular area surrounding the pressure openings. This permits a radial supply of the suction gas into the cylinder head. Before entering the suction chamber, this sucked-in gas is to be used for cooling purposes.

GB 2 083 566 A shows a further design of a piston compressor with several centrally arranged pressure openings. The pressure openings are covered by a common valve element in the form of a ring. A plurality of suction openings is provided in an annular area, which surrounds the pressure openings.

U.S. Pat. No. 5,173,040 shows an air compressor, in which several suction openings with a common suction valve element are located in one half of the cross-sectional face of the compression chamber, whereas several pressure openings are located in the other half.

U.S. Pat. No. 3,926,214 shows a similar embodiment, in which several groups of suction openings are provided. The suction openings of one group are covered by a common valve element in the form of a band, which is supported at both ends. One end is fixed and the other end is movable.

### BRIEF SUMMARY OF THE INVENTION

The invention is based on the task of providing a piston compressor with a good efficiency.

With a piston compressor as mentioned in the introduction, this task is solved in that the suction valve arrangement opens into the compression chamber in a central area of the cylinder and the pressure valve arrangement has a plurality of pressure openings, which are located in an annular area surrounding the suction valve arrangement.

With this embodiment, the gas is sucked into the compression chamber in the area of the cylinder axis. This substantially reduces and timely delays the direct contact of the cold refrigerant gas with the hot cylinder wall. This means that during the suction phase, that is, during the expansion of the compression chamber, the suction gas is not too strongly heated and expanded, as is the case, when the gas is supplied in the immediate vicinity of the cylinder wall. Thus, during each suction process a larger mass flow can be sucked in. This improves the efficiency of the compressor. The discharge of the compressed gas then occurs in an area, which is closer to the cylinder wall. The fact that several pressure openings are available causes a favourable design of the flow conditions for the discharge of the larger refrigerant mass. The total sum of the cross-sections of the pressure valve openings can be chosen to be larger than the cross-section of a single opening as mostly used until now. The plurality of pressure openings ensures that the increased mass flow cannot only be compressed but also be discharged without large obstacles.

Preferably, the suction valve arrangement has a twist arrangement, which provides a suction gas flow with a movement component in the circumferential direction of the cylinder. Thus, the sucked-in gas is brought to rotate, so that it forms an inlet flowleading the gas into the cylinder from the cylinder axis in a star-shaped and downwards inclined direction. Thus, a contact with the cylinder wall only occurs relatively late. On the other hand, the twist or rotation movement also causes that a centrifugal force acts upon the gas. Then, the gas can distribute from the centrically arranged suction valve arrangement in the compression chamber so that a good filling is achieved.

Preferably, the suction valve arrangement has several suction openings, each being covered by its own suction valve element. This embodiment firstly has the advantage that during the suction stroke a relatively large cross-sectional area is available, through which the gas can flow into the compression chamber. The fact that each suction opening has its own suction valve element causes that the mass of a suction valve element can be kept small. Accordingly, a fast reaction is possible. Also the stroke to be performed by the individual suction element to release the suction opening can be kept small.

It is preferred that the suction valve elements open in the circumferential direction. Thus, the suction valve elements form the twist arrangement or at least part of it. The fact that the suction valve elements open in the circumferential direction causes that the gas flow flowing into the compression chamber is made rotating, meaning that the suction valve elements cause the rotational movement of the gas flow.

It is preferred that each suction valve element opens in the direction of an area, in which the rear of a neighbouring suction valve element is located. Thus, the gas flow passing a suction opening is firstly redirected in the circumferential direction by the suction valve element covering the suction opening in question. After this redirecting, the gas flow meets the rear of the neighbouring suction valve element. As the neighbouring suction valve element is also open and therefore slightly inclined, the gas flow is slightly deflected again. Thus, as mentioned above, the gas flow gets a direction, which is directed downwards into the compression chamber in an inclined manner.

Preferably, the suction valve elements surround each other, at least partly. Thus, the suction valve elements are interlaced with each other. When opening during a suction stroke of the piston, the suction valve elements are, in a manner of speaking, moving helically into the cylinder, thus forming a guiding device, which leads to the above mentioned rotational movement of the gas flow.

In an alternative embodiment it is ensured that the suction valve has a suction valve element, which, during an opening movement, is displaced into the compression chamber parallel to the level of the suction valve plate. With this embodiment of the suction valve, a rotation effect does not occur. However, a fast release of a relatively large opening is achieved, namely a gap between the suction valve element and the suction valve plate, which practically extends all the way round the complete suction valve element. Thus, the area of the cylinder available for the suction inlet is utilised to an optimum.

It is preferred that the suction valve element is located centrally on the suction valve plate. Thus, the cylinder is filled uniformly from a central area.

Preferably, the suction valve element is connected with the suction valve plate by at least one holding arm, which, in the closed state, surrounds the suction valve element at least partly in the circumferential direction. On the one hand this holding arm permits a relatively fast opening movement. On the other hand, this holding arm forms no large resistance to the incoming gas, so that the filling of the cylinder can take place relatively quickly.

It is preferred that the holding arm surrounds the suction valve element over at least 180°. When several holding arms are provided, this circumferential angle is distributed on the several holding arms. At any rate, the circumferential angle can also amount to more than 180°. The fact that the holding arms are led around the suction valve element in the circumferential direction causes that a relatively large length occurs, so that a sufficient opening stroke of the suction valve element can be realised.

Preferably, at least three holding arms are provided, which partly overlap in the circumferential direction. With such an embodiment, it is ensured that also during an opening movement the suction valve element remains practically in the centre of the suction valve plate.

Preferably, the pressure openings end in an annular channel. In the annular channel, the discharged gas can then flow to an outlet. The annular channel can be dimensioned so that it gives as little flow resistance to the discharged gas as possible.

It is preferred that each pressure opening is covered by a pressure valve element, which opens in the circumferential direction. Thus, the pressure valve element ensures that the gas flowing out of the pressure opening concerned gets the right direction, namely the circular direction of the annular channel. With a bottom or cover wall, the annular channel can also form a stop for the movement of the pressure valve elements.

It is also advantageous, when the pressure valve element and the suction valve element open in the same circumferential direction. Thus, the rotation movement of the refrigerant gas caused by the suction valve arrangement can be even better utilised. The kinetic energy of the refrigerant gas is at least partly maintained, so that the efficiency of the compressor can be improved.

It is preferred that all pressure valve elements open in the same circumferential direction. The gas flow, which is discharged through the pressure openings, will then in total have a direction in the circumferential direction without causing the occurrence of unnecessary eddy flows, when two gas flows discharged from different pressure openings get in contact with each other.

It is also advantageous that the pressure valve elements are made as flexible tongues originating from a pressure valve plate, each tongue being located in a recess, which extends over the tongue in the radial and/or the circumferential direction. Thus, with opened pressure valve element, these recesses only cause a relatively small flow resistance to the discharged gas.

Preferably, the annular channel extends over the front side of the cylinder, and the pressure openings are located very close to the circumferential wall of the cylinder. In the extreme case this means that in the radial direction the pressure openings end with the inner wall of the cylinder. In practice, however, small deviations will exist here. This embodiment has several advantages. Particularly in connection with the rotational movement of the gas flow, which also continues in the compression phase, the discharged gas already tends to flowing radially outwards and thus to the wall of the cylinder. Thus, the pressure openings are already in the right place, that is, where the gas will flow to. In the annular channel sufficient space will then be available to adopt the gas discharged by the pressure openings. In the suction phase, however, this causes that the pressure in the annular channel will be higher than the pressure in the compression chamber. With this higher pressure there is a risk that the valve plate arrangement will bend. This risk is considerably reduced in that on the side facing the compression chamber the valve plate arrangement is at least partly supported by the cylinder. The forces acting upon the valve plate arrangement are thus partly adopted by the wall of the cylinder.

Preferably, the valve plate arrangement has a base plate, a suction valve plate and a pressure valve plate, the suction valve plate and the pressure valve plate being located on the side of the base plate facing the cylinder, the pressure valve plate forming at least one valve seat for the suction valve arrangement and the suction valve plate forming at least one valve seat for the pressure valve arrangement. As the pressure valve plate and the suction valve plate, which are usually made of spring steel, can be kept substantially thinner than the base plate, which must provide a certain mechanical stability, this contributes to keeping the harmful space or the dead space very small. This further improves the efficiency of the compressor.

Preferably, the suction valve plate, the pressure valve plate and the base plate are connected with each other by at least one circumferential, gas-tight annular joint, which penetrates the suction valve plate and the pressure valve plate and separates the suction valve arrangement from the pressure valve arrangement. Thus, the annular joint has two tasks. On the one side, it connects the suction valve plate, the pressure valve plate and the base plate. On the other hand, it seals the suction area against the pressure area, so that neither during a suction stroke nor during a pressure stroke gas can pass by the suction valves or pressure valves into the respective other area. Also this improves the efficiency of the compressor. The annular joint can, for example, be made by means of welding, soldering or gluing.

Preferably, the annular joint forms a bead projecting into the compression chamber, and in the area of the bead the piston has a recess in its front side. This simplifies the manufacturing. The bead occurring during the manufacturing of the annular joint does not have to be removed. Under certain circumstances, this could also cause a weakening of the connection inside the valve plate arrangement. However, the dead space remains small, as the bead can enter into the piston.

Preferably, at least one second annular joint is provided, which extends radially outside the pressure openings. Also this annular joint can connect the suction valve plate, the pressure valve plate and the base plate with each other and penetrate the suction valve plate and the pressure valve plate. The second annular joint ensures that the valve plate arrangement is tight radially outwards, so that no additional measures for a tightening radially outwards have to be taken.

Preferably, the second annular joint is located in the area of a sealing, which is located between the front side of the cylinder and the valve plate arrangement. The second annular joint, which usually also forms a bead, then presses somewhat into the sealing. This has the advantage that in the radial direction the sealing is held by the annular joint, so that also with higher pressures a displacement must not be feared. On the other hand, no measures are required for handling the bead of the second annular joint.

Preferably, the base plate has a circumferential flange, which extends in the axial direction and forms a recess, in which a front side of the cylinder is inserted. Thus, the cylinder is held reliably on the valve plate arrangement in the circumferential direction.

It is preferred that in the circumferential direction the flange has at least one interstice, in which a radial extension engages, which is formed on the suction valve plate and/or the pressure valve plate. This extension can be used for correct angular positioning of the suction valve plate and/or the pressure valve plate.

### BRIEF DESCRIPTION OF THE DRAWINGS

In the following, the invention is described by means of a preferred embodiment in connection with the drawings, showing:

FIG. 1 is a section of a piston compressor;

FIG. 2 is a suction valve plate seen from the compression chamber;

FIG. 3 is a perspective view of the suction valve plate according to FIG. 2;

FIG. 4 is a pressure valve plate seen from the compression chamber;

FIG. 5 is a top view of a valve system seen from the compression chamber;

FIG. 6 is a base plate seen from the compression chamber;

FIG. 7 is the base plate seen from the opposite side;

FIG. 8 is a side view of the base plate; and

FIG. 9 is various embodiments of modified suction valve plates.

### DETAILED DESCRIPTION OF THE INVENTION

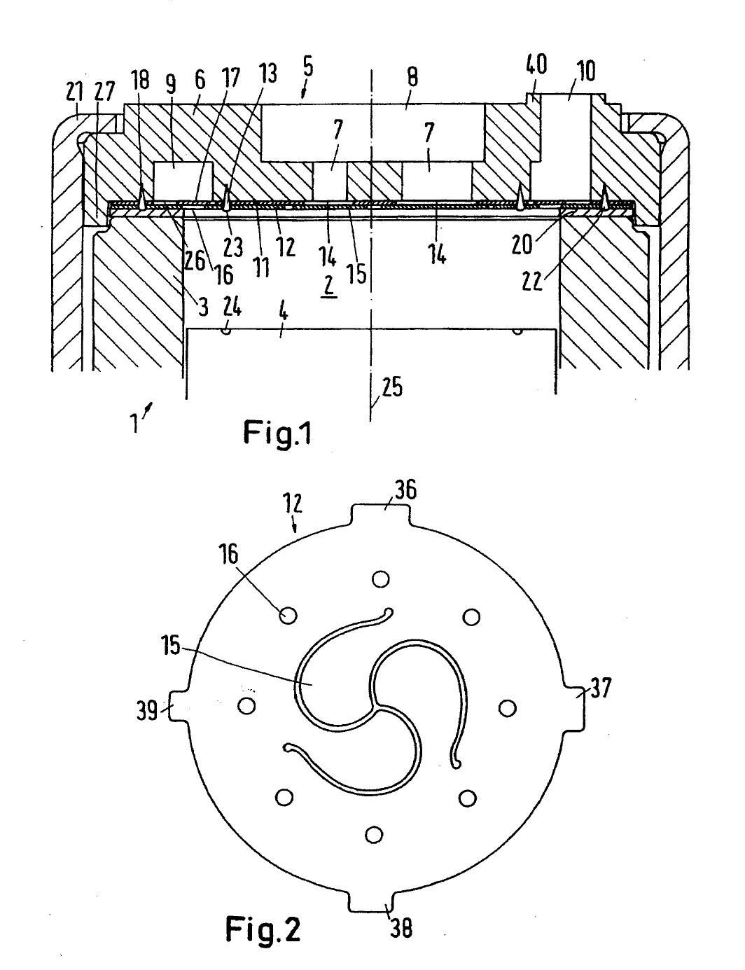

A piston compressor 1, only shown in sections in FIG. 1, has a compression chamber 2, which is bordered by a cylinder 3, a piston 4 and a valve plate arrangement 5. When the piston 4 reciprocates in the cylinder 3, the size of the compression chamber changes. When the piston 4 is moved away from the valve plate arrangement 5, the compression chamber 2 expands, and gas, for example a refrigerant gas, is sucked in. When the piston 4 is moved towards the valve plate arrangement 5, the gas in the compression chamber 2 is compressed and finally discharged.

For the control of the gas flows, the valve plate arrangement 5 has a base plate 6, which is relatively massive, and provides the valve plate arrangement 5 with the largest share of its mechanical stability. The base plate 6 has several suction channels 7, which originate from a recess 8. One purpose of the recess 8 is the mounting of a suction muffler, not shown in detail.

Further, the base plate 6 has an annular channel 9, which his connected with a pressure connection 10.

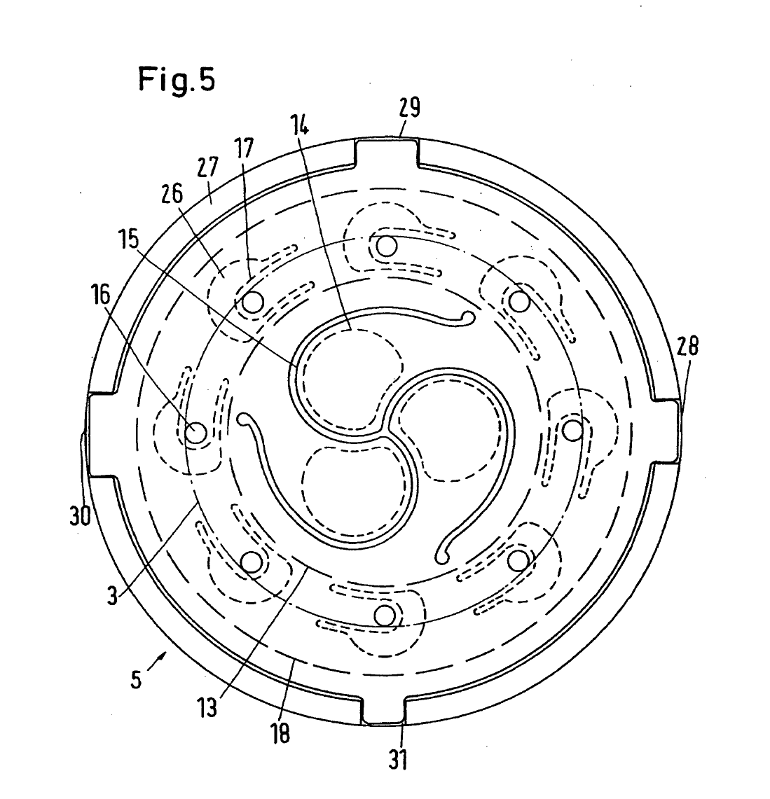

On the side of the base plate 6 facing the compression chamber 2 is located a pressure valve plate 11 and on the side of the pressure valve plate 11 facing the compression chamber 2 is located a suction valve plate 12. The suction valve plate 12, the pressure valve plate 11 and the base plate 6 are connected with each other by means of an annularly shaped welding joint 13. The welding joint 13 penetrates the pressure valve plate 11 and the suction valve plate 12. As shown in FIG. 5, it is closed in the circumferential direction and forms, as will be explained in detail later, a block between a pressure valve area and a suction valve area.

The pressure valve plate 11 has several suction openings 14, the number and the positions of the suction openings 14 corresponding to the number and positions of the suction channels 7. Each suction opening 14 is somewhat larger than the belonging suction channel 7. The suction openings 14 are covered by suction valve elements 15. The suction valve elements 15 are part of the suction valve plate 12.

The suction valve plate 12 has a plurality of pressure openings 16, each being covered by a pressure valve element 17. The pressure valve elements 17 are part of the pressure valve plate 11.

The base plate 6, the pressure valve plate 11 and the suction valve plate 12 are connected with each other by means of a second welding joint 18, which is located radially outside the annular channel 9. Also the second welding joint 18 is continuous in the circumferential direction and forms a gas-tight connection.

Together, the base plate 6, the pressure valve plate 11 and the suction valve plate 12 form the valve plate arrangement 5 (FIG. 5), which is mounted on the front side of the cylinder 3 by way of a sealing 20. By means of a merely schematically shown clip 21, the valve plate arrangement 5 can be connected with the cylinder 3. Here, the second welding joint 18 can have a bead 22 projecting in the direction of the cylinder 3 and pressing somewhat against the sealing 20. When the first welding joint 13 also has a bead 23, it is expedient to provide the front side of the piston 4 with a circumferential recess 24, into which the bead 23 can enter in the upper dead point of the piston 4 to keep the dead space as small as possible.

Preferably, the pressure valve plate 11 and the suction valve plate 12 are made of spring steel and undetachably connected with each other and with the base plate 6 by the welding joints 13, 18. The suction valve elements 15 and the pressure valve elements 17 are made by tongues, which are formed in the suction valve plate 12 and the pressure valve plate 11 by means of punching. With the edge of the pressure opening 16, the suction valve plate 12 forms valve seats for the pressure valve arrangement. With the edge of the suction openings 14, the pressure valve plate 11 forms valve seats for the suction valve arrangement.

The suction openings 14 are provided in the central area of the pressure valve plate 11. Here, the suction valve elements 15 are made as interlaced parts, which partly surround each other. The thickness of the suction valve plate 12 has been chosen so that a suction stroke provides a sufficient, but not excessive deflection of the suction valve elements 15, as here no stop device for the suction valve elements 15 is available. During their opening movement, the pressure valve elements 17, however, can come to bear on the base plate 6 in the annular channel 9.

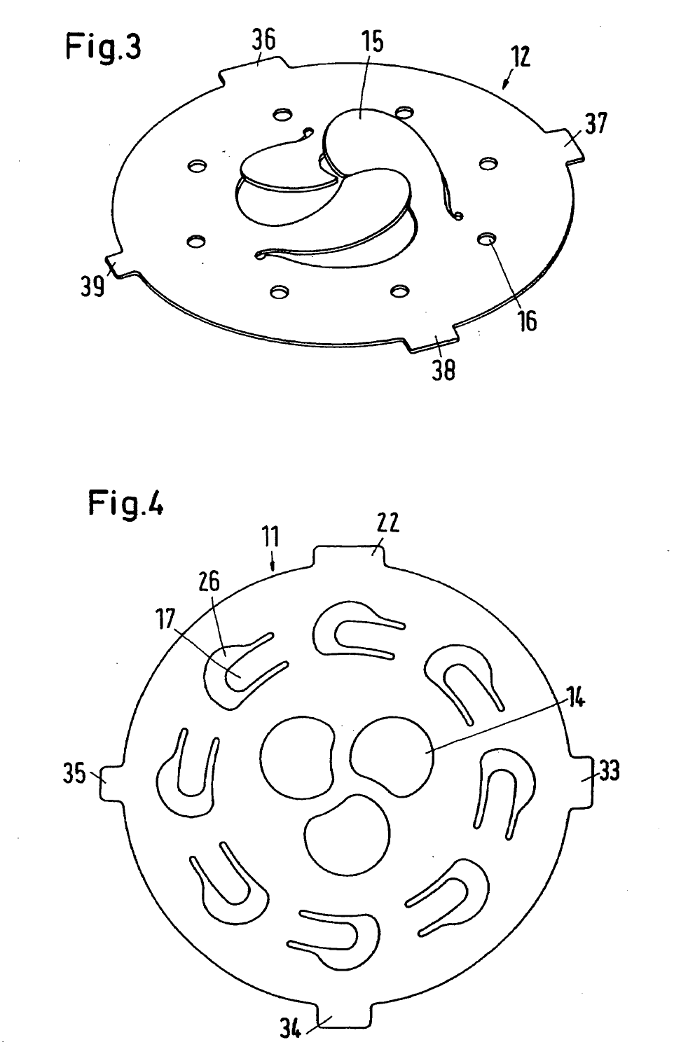

As can be seen, particularly from FIG. 3, the suction valve elements 15 open helically into the compression chamber 2 during a suction stroke. Thus, each suction valve element 15 opens so that a movement component of the gas deflected by the suction valve element 15 has a large component in the circumferential direction (in relation to a merely schematically shown axis 25 of the cylinder 3). Thus, the gas flow deflected by a suction valve element 15 reaches the rear (that is, the side facing the compression chamber 2) of the neighbouring suction valve element 15. This gives the gas flow a direction, in which the gas is led into the compression chamber 2 from the cylinder axis 25 in a star shaped and downwards inclined direction. Thus, a contact with the cylinder wall only takes place relatively late. The fact that circumferentially all suction valve elements 15 open in the same direction causes that after the meeting with the inner wall of the cylinder 3 an overall rotating gas flow appears.

The pressure openings 16 are arranged so that their radial outer extension practically ends with the inner wall of the cylinder 3. However, the annular channel 9 continues extending radially outwards. This causes that during a suction stroke the suction valve plate 12 is sufficiently supported by the front side of the cylinder 3, so that the relatively short area, which remains between the wall of the cylinder 3 and the welding joint 23, cannot bend.

The fact that the pressure openings 16 are located at the radial outer area of the compression chamber 2 results in favourable conditions during the discharge of the gas from the compression chamber 2. As the gas flow rotates, a centrifugal force acts upon the gas, which moves it in the direction of the cylinder 3 wall. Thus, the gas is directed radially in the direction of the pressure openings 16.

As can be seen particularly from FIG. 4, each pressure valve element 17 is located in a recess 26, which extends radially outwards and which extends over the pressure valve element 17 in the circumferential direction. This gives favourable flow conditions. When the pressure valve element 17 opens, a larger flow cross-section, that is, more space, is available for the gas flow. As can be seen from FIG. 1, the recess 26 can extend in the radial direction right up to the radially outer border of the annular channel 9.

As can be seen from FIG. 4, all pressure valve elements 17 point in the same direction. When, during a compression stroke of the piston 4, the compression chamber 2 is reduced and gas is discharged, the gas flow will be deflected in the circumferential direction by the pressure valve elements 17. In other words, the gas is led by the pressure valve elements 17, which are made as tongue-shaped leaf springs, in such a manner that a gas flow occurs in the annular channel 9, which only flows in one direction. Thus, the maximum deflection of the gas flow will be 90°, before it can be discharged from the annular channel 9 through the pressure connection 10.

It is, of course, particularly expedient, when the pressure valve elements 17 direct the gas in the same direction, in which it rotates anyway, when it has been caused to rotate by the suction valve elements 15. The rotational movement of the gas namely also continues during the compression stroke of the piston 4. Thus, the kinetic energy of the gas is at least partly maintained and can then be used to improve the efficiency of the compressor. Originally, the centric entry of the gas into the compression chamber 2 together with the rotational movement was initially meant to keep the gas away from the hot cylinder 3 wall for as long as possible.

As can be seen from FIGS. 1 and 5, the base plate 6 has a circumferential flange 27, by means of which the cylinder 3 can be positioned in relation to the base plate 6. In FIG. 5 the valve plate arrangement 5 is shown as a view from the compression chamber 2. All elements, which are covered by the pressure valve plate 12, are shown with dotted lines.

It can be seen that distributed in the circumferential direction the flange adopts four recesses 28 to 31, of which the recess 31 is clearly smaller in the circumferential direction than the remaining recesses 28 to 30. Both the pressure valve plate 11 and the suction valve plate 12 have corresponding projections 32 to 35 or 36 to 39, respectively, which project radially and which can engage in the recesses 28 to 31, so that the pressure valve plate 11 and the suction valve plate 12 can be positioned at a predetermined angular position in relation to the base plate 6.

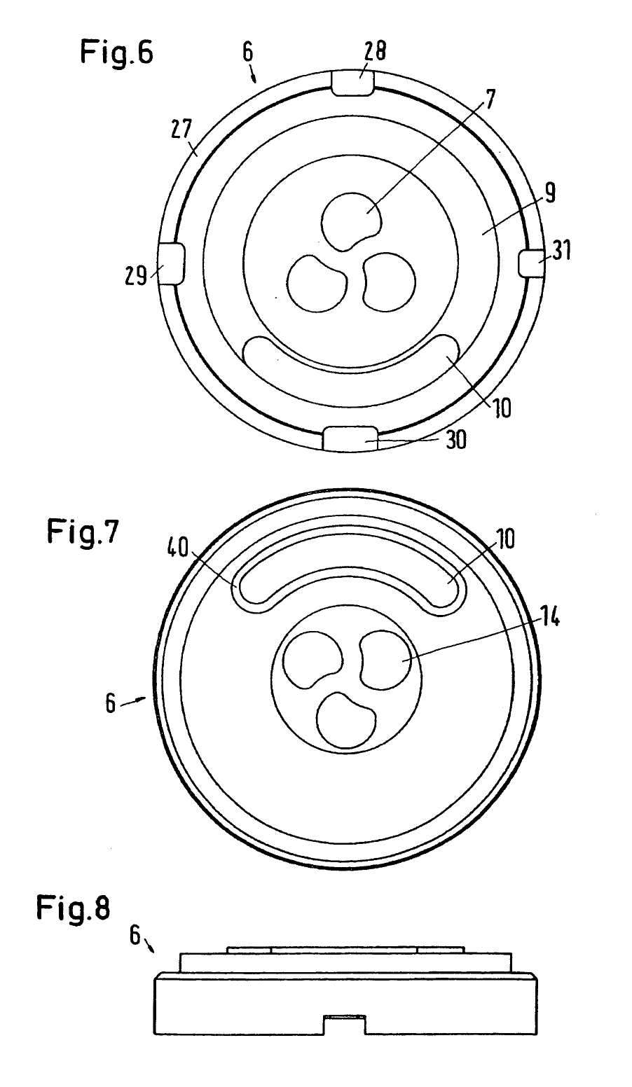

As can be seen particularly from FIGS. 6 and 7, the pressure connection 10 has a kidney-shape. It is surrounded by a projection 40, so that here a pressure muffler can be inserted and fixed.

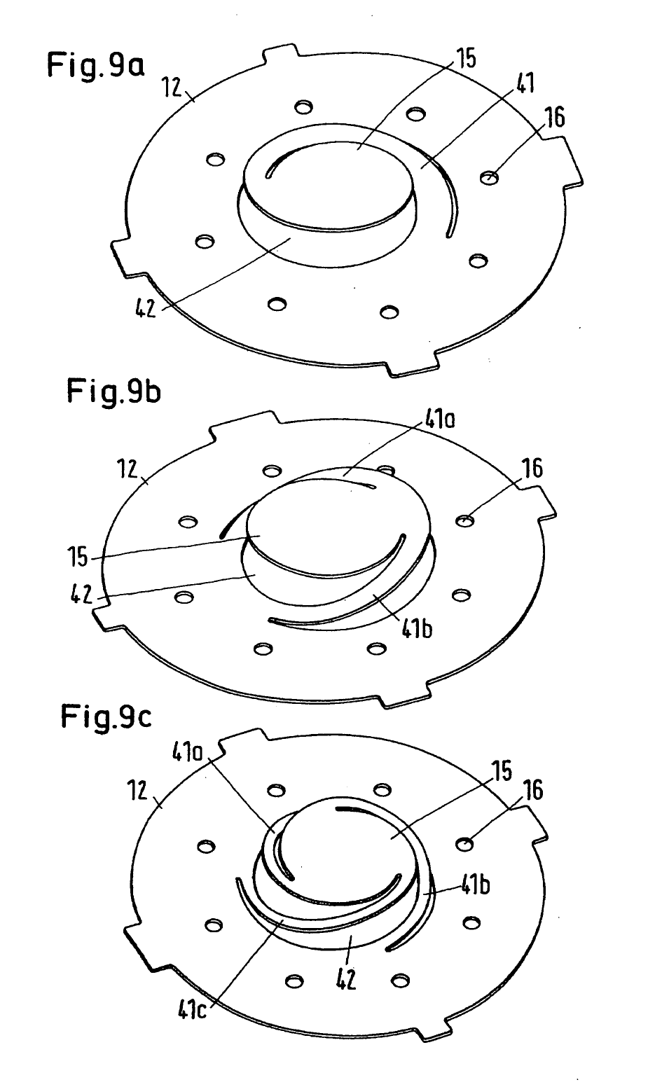

FIG. 9 shows examples of modified suction valve plates. Same and functionally equal elements have the same reference numbers as in the FIGS. 1 to 3.

In the embodiment according to FIG. 9a the suction valve element 15 is connected with the suction valve plate 12 via one single holding arm 41. In the closed state the holding arm 41 surrounds the suction valve element over an angle of approximately 200°. In this connection, it is led around the suction valve element 15 in the circumferential direction. The suction valve element 15 is provided in a central section of the suction valve plate 12 and covers the suction opening in the closed state, which is not shown. During the opening movement, the suction valve element 15 is displaced into the compression chamber in parallel with the level of the suction valve plate. This gives an annular gap 42, through which the gas can enter. Thus, the area of the cylinder available for the suction inlet is utilised optimally.

FIG. 9b shows a modified embodiment with two holding arms 41a, 41b, engaging sides of the suction valve element 15, and being located opposite each other. Shown is an embodiment, in which the two holding arms 41a, 41b are bending in the same direction, when the suction valve element 15 lifts off from the suction opening, which is not shown in detail. However, it is also possible for the two holding arms 41a, 41b to tilt in different directions. In this case, the suction valve element 15 would turn somewhat during opening, exactly like in the embodiment according to FIG. 9a.

FIG. 9c shows a third embodiment, in which the suction valve element 15 is connected with the suction valve plate by means of three holding arms 41a, 41b, 41c. Also here a gap 42 occurs, when the suction valve element 15 is moved from the level of the suction valve plate 12.

As, in a manner of speaking, the narrow sides of the holding arms 41a, 41b, 41c are exposed to the gas flow, they create no significant resistance to this gas flow.

All three embodiments provide suction valves enabling a substantially uniform inlet of suction gas over the whole circumference. Compared to unilaterally opening valves this gives more favourable flow conditions and a fast and uniform filling of the cylinder.

In the embodiment according to FIG. 9c, the holding arms 41a, 41b, 41c overlap each other somewhat in the circumferential direction. This has the advantageous effect that the holding arms 41a, 41b, 41c can be made relatively long in the circumferential direction. Longer holding arms 41a, 41b, 41c are more easily deformed and permit a larger opening stroke of the suction valve element 15.

While the present invention has been illustrated and described with respect to a particular embodiment thereof, it should be appreciated by those of ordinary skill in the art that various modifications to this invention may be made without departing from the spirit and scope of the present invention.

1. A piston compressor, particularly a hermetical refrigerant compressor, comprising:
a compression chamber, which is bordered by a cylinder, a piston and a valve plate arrangement, which has a suction valve arrangement and a pressure valve arrangement,
wherein the suction valve arrangement opens into the compression chamber in a central area of the cylinder and the pressure valve arrangement has a plurality of pressure openings, which are located in an annular area surrounding the suction valve arrangement.

2. The piston compressor according to claim 1, wherein the suction valve arrangement has a twist arrangement, which provides a suction gas flow with a movement component in the circumferential direction of the cylinder.

3. The piston compressor according to claim 1, wherein the suction valve arrangement has several suction openings, each being covered by its own suction valve element.

4. The piston compressor according to claim 3, wherein the suction valve elements open in the circumferential direction.

5. The piston compressor according to claim 4, wherein each suction valve element opens in the direction of an area, in which the rear of a neighboring suction valve element is located.

6. The piston compressor according to claim 3, wherein the suction valve elements surround each other, at least partly.

7. The piston compressor according to claim 1, wherein the suction valve has a suction valve element, which, during an opening movement, is displaced into the compression chamber parallel to the level of the suction valve plate.

8. The piston compressor according to claim 7, wherein the suction valve element is located centrally on the suction valve plate.

9. The piston compressor according to claim 7, wherein the suction valve element is connected with the suction valve plate by at least one holding arm, which, in the dosed state, surrounds the suction valve element at least partly in the circumferential direction.

10. The piston compressor according to claim 9, wherein the holding arm surrounds the suction valve element over at least 180°.

11. The piston compressor according to claim 10, wherein at least three holding arms are provided, which partly overlap in the circumferential direction.

12. The piston compressor according claim 1, wherein the pressure openings end in an annular channel.

13. The piston compressor according to claim 12, wherein each pressure opening is covered by a pressure valve element, which opens in the circumferential direction.

14. The piston compressor according to claim 13, wherein the pressure valve element and the suction valve element open in the same circumferential direction.

15. The piston compressor according to claim 13, wherein all pressure valve elements open in the same circumferential direction.

16. The piston compressor according to claim 13, wherein the pressure valve elements are made as flexible tongues originating from a pressure valve plate, each tongue being located in a recess, which extends over the tongue in the radial and/or the circumferential direction.

17. The piston compressor according to claim 12, wherein the annular channel extends over the front side of the cylinder, and the pressure openings are located very dose to the circumferential wall of the cylinder.

18. The piston compressor according to claim 1, wherein the valve plate arrangement has a base plate, a suction valve plate and a pressure valve plate, the suction valve plate and the pressure valve plate being located on the side of the base plate facing the cylinder, the pressure valve plate forming at least one valve seat for the suction valve arrangement and the suction valve plate forming at least one valve seat for the pressure valve arrangement.

19. The piston compressor according to claim 18, wherein the suction valve plate, the pressure valve plate and the base plate are connected with each other by at least one circumferential, gas-tight annular joint, which penetrates the suction valve plate and the pressure valve plate and separates the suction valve arrangement from the pressure valve arrangement.

20. The piston compressor according to claim 19, wherein the annular joint forms a bead projecting into the compression chamber, and in the area of the bead the piston has a recess in its front side.

21. The piston compressor according to claim 19, wherein at least one second annular joint is provided, which extends radially outside the pressure openings.

22. The piston compressor according to claim 21, wherein the second annular joint is located in the area of a sealing, which is located between the front side of the cylinder and the valve plate arrangement.

23. The piston compressor according to claim 1, wherein the base plate has a circumferential flange, which extends in the axial direction and forms a recess, in which a front side of the cylinder is inserted.

24. The piston compressor according to claim 23, wherein in the circumferential direction the flange has at least one interstice, in which a radial extension engages, which is formed on the suction valve plate and/or the pressure valve plate.
